(19) United States
(12) Patent Application Publication
(10) Pub. No.: US 2022/0218514 A1
(43) Pub. Date: Jul. 14, 2022

(54) OPHTHALMIC DEVICES, SYSTEMS AND METHODS FOR TREATING DRY EYE

(71) Applicant: Verily Life Sciences LLC, South San Francisco, CA (US)

(72) Inventors: Dimitri Azar, South San Francisco, CA (US); Fred Shungneng Lee, South San Francisco, CA (US)

(21) Appl. No.: 17/610,641

(22) PCT Filed: May 14, 2020
(86) PCT No.: PCT/US2020/032816

§ 371 Date: Nov. 11, 2021

Related U.S. Application Data
(60) Provisional application No. 62/847,716, filed on May 14, 2019.

(51) Int. Cl. A61F 7/00

(52) U.S. Cl. CPC A61F 7/007; A61F 2007/0075; H02J 50/10; A61F 2007/0077; A61F 2007/0004

(57) ABSTRACT

A system for treating dry eye is presented. According to some aspects, the system includes an underlid device having an anterior surface and a posterior surface, wherein the anterior surface is configured to contact a portion of an eyelid, and wherein the posterior surface is configured to contact a portion of an eyeball. The underlid device further includes a Peltier heat pump. The Peltier heat pump includes a first surface configured to heat the eyelid when the device is positioned between the eyelid and the eyeball, and a second surface configured to cool a portion of a surface of the eyeball when the device is positioned between the eyelid and the eyeball. The underlid device further includes an energy storage element coupled to the Peltier heat pump and configured to supply power to the Peltier heat pump.

### TECHNICAL FIELD

The present disclosure relates generally to ophthalmic devices for treating dry eye, and, in particular but not exclusively, relates to ophthalmic devices for treating dry eye through heating Meibomian glands and/or cooling a surface of the eye.

### BACKGROUND

Dry eye disease is one of the most common eye conditions worldwide. Many dry eye cases are related to the absence of, or severely reduced production of, meibum, an oily substance produced by the Meibomian (also expressed as meibomian) glands. There are Meibomian glands located in the upper and lower eyelids, and lipids are a major component of meibum. One of the root causes of the lack of meibum are Meibomian glands that become completely or partially clogged. Lack of meibum production and secretion are some symptoms of Meibomian gland dysfunction.

Many dry eye cases are also related to lack of tear production or tears that evaporate too quickly.

Normally, the lipid layer produced by the Meibomian glands spreads evenly into a thin protective film over the air-tear interface above the cornea. Every time a person blinks a slight amount of lipid protective film is spread. However, there are many conditions under which this oily layer no longer spreads out evenly over the tear film and this process can be interrupted, reduced, or even stopped entirely, including inadequate blinking from excessive screen time known as computer vision syndrome.

The absence of an outer protective lipid layer reduces the evaporation time for the tear film covering the eye leading to interrelated issues of inadequate production of tears and meibum.

Some treatments for Meibomian gland dysfunction include using warm compresses, eyelid cleansing compounds, and massaging the eyelids gently to try to reduce eyelid inflammation. Other known eye treatments include heating the outside of the eyelids using heating pads. Some treatments for inadequate tear production include application of various types of artificial tears. Known treatments either lack effectiveness or are too costly.

Thus, there remains a need for less costly and more convenient ways of treating dry eye disease generally, and Meibomian gland dysfunction and/or inadequate tear production in particular.

### SUMMARY

The present disclosure advantageously describes a system for treating dry eye. According to some aspects, the system includes an underlid device having an anterior surface and a posterior surface, wherein the anterior surface is configured to contact a portion of an eyelid, and wherein the posterior surface is configured to contact a portion of an eyeball. The underlid device further includes a Peltier heat pump. The Peltier heat pump includes a first surface configured to heat the eyelid when the underlid device is positioned between the eyelid and the eyeball, and a second surface configured to cool a portion of a surface of the eyeball when the underlid device is positioned between the eyelid and the eyeball. The underlid device further includes an energy storage element coupled to the Peltier heat pump and configured to supply power to the Peltier heat pump. Some embodiments further include a buck converter configured to couple the energy storage element to the Peltier heat pump.

In some aspects, the present disclosure describes a device for treating dry eye and configured to be positioned between an eyelid and sclera. The device includes a first surface that is convex for contacting the eyelid and a second surface that is concave for contacting the sclera. The device further includes an energy storage device and a Peltier device. The Peltier device includes a third surface configured to heat a portion of the eyelid and a fourth surface configured to cool a portion of the sclera when power is delivered to the Peltier device. The device further includes a circuit configured to couple the energy storage device to the Peltier device to supply power to the Peltier device, wherein heat is transferred within the Peltier device when power is supplied to the Peltier device resulting in heating of the third surface and cooling of the fourth surface, thereby heating the portion of the eyelid and cooling the portion of the sclera, respectively. In some embodiments, the circuit includes a buck converter.

In some aspects, the present disclosure describes a method of ophthalmic treatment using an underlid device when the underlid device is positioned beneath the surface of an eyelid. The underlid device includes an energy storage element, a buck converter and a Peltier device connected in series. The Peltier device includes a first surface and a second surface, wherein the first surface is configured to heat a portion of the conjunctiva of the eyelid and the second surface is configured to cool a portion of the sclera. The Peltier device is configured to transfer heat such that the first surface increases in temperature and the second surface decreases in temperature when power is supplied using the energy storage element and the buck converter. The method includes generating a signal, by a sensor, determining that a condition is satisfied based on the signal, and based on the condition being satisfied, supply power to the Peltier device using the energy storage element and the buck converter to heat the first surface and cool the second surface, thereby heating the portion of the conjunctiva and cooling the portion of the sclera.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIGS. 1A and 1B present different views of a system for treating dry eye, according to some aspects of the present disclosure.

FIG. 2 is a block diagram of an embodiment of an underlid device, according to some aspects of the present disclosure.

FIG. 3 is a circuit diagram of an embodiment of a buck converter in an underlid device, according to some aspects of the present disclosure.

FIGS. 4A and 4B are perspective views of portions of different embodiments of underlid devices that include a Peltier device, according to some aspects of the present disclosure.

FIG. 5 is a block diagram of components in a frame, according to some aspects of the present disclosure.

FIG. 6 is a view of an eyelid side of an underlid device, according to some aspects of the present disclosure.

FIG. 7 presents a method of operating an underlid device, according to some aspects of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. For example, while therapeutic devices are illustrated in terms of devices placed underneath a lower eyelid for treatment of dry eye, the devices can also be placed underneath an upper eyelid for treatment of dry eye. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

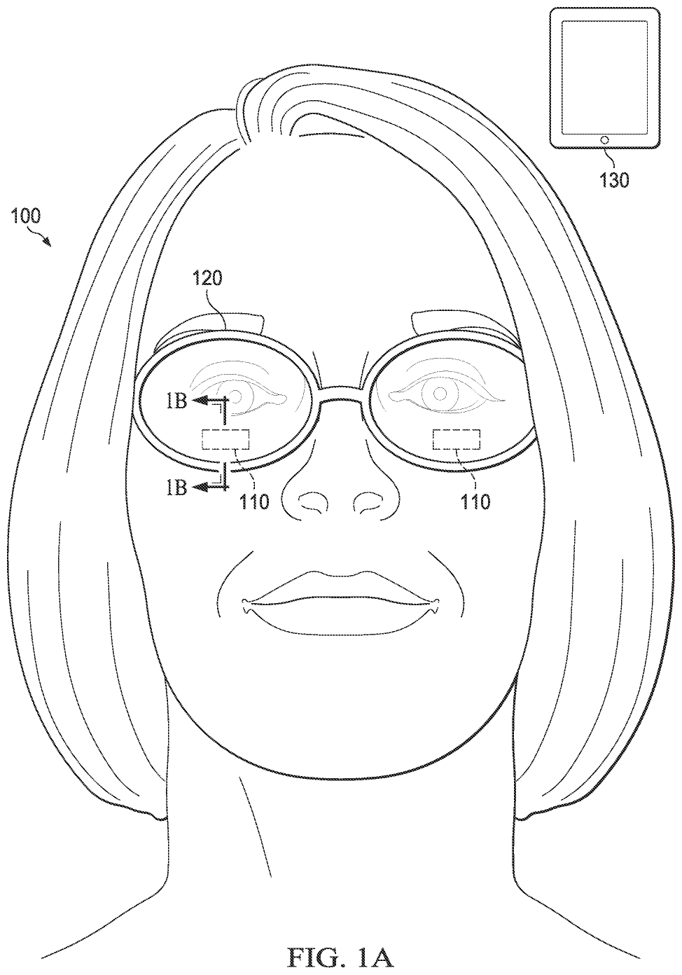

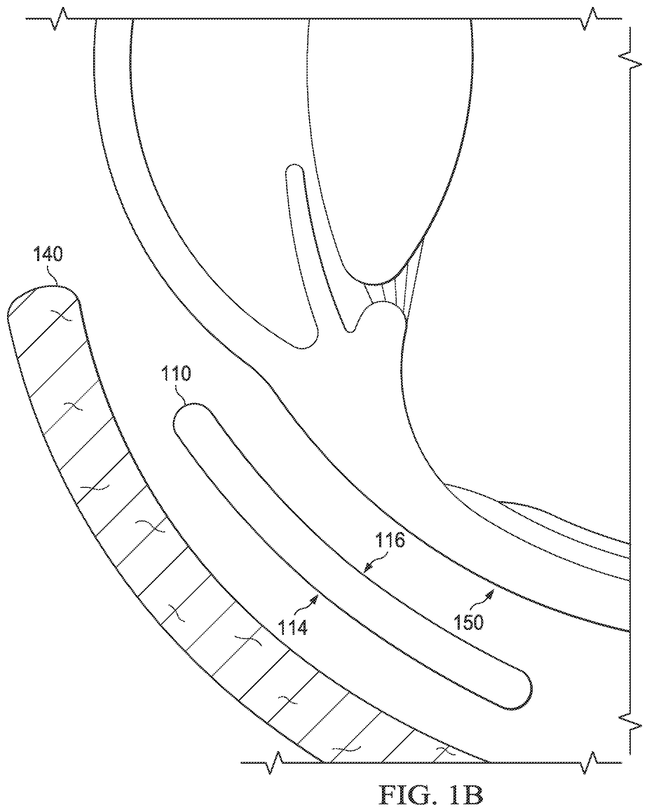

An exemplary system 100 for treating dry eye according to one embodiment is illustrated in FIG. 1A. As shown, the system 100 includes a pair of devices 110, each device 110 positioned between a lower eyelid and a patient's eyeball, a frame 120 worn by the patient, and a communication device 130, such as a smartphone, cellular phone or tablet. FIG. 1B is a cross-sectional side view of device 110 positioned between an eyelid 140 and eyeball 150, with the cross-section along the line indicated in FIG. 1A. FIG. 1B illustrates the eyelid 140 and eyeball 150 spaced apart from the device 110, but in use the device 110 will typically touch at least a portion of eyeball 150 and at least a portion of eyelid 140.

As shown in FIG. 1A, each device 110 is configured to cover only a portion of the surface of an eyeball residing underneath an eyelid when the eyelid is open and not cover any portion of the pupil. Thus, the form factor of each device is significantly different than a contact lens, which is typically designed to cover the entire pupil when worn. In other words, in some embodiments, each device 110 is configured to reside against only the sclera. Although each device 110 is illustrated as residing underneath a lower eyelid, each device 110 may instead reside underneath an upper eyelid for treatment of dry eye.

Each device 110 is located underneath an eyelid and adjacent to an inner surface of the eyelid, as illustrated in FIG. 1B, and an outline of a top view of each device 110 as projected on an outer surface of a patient's skin is shown in FIG. 1A. In some embodiments, each device 110 may be referred to as an underlid device 110. Although two devices 110 are shown in FIG. 1A, a patient may only wear one device 110 at a given time. In an embodiment, each device 110 includes a Peltier device (not shown), sometimes referred to as a Peltier heat pump, as discussed further herein.

The device 110 comprises a first surface (or anterior surface) 114 configured to heat the eyelid when the device is positioned underneath the eyelid. Correspondingly, the device 110 comprises a second surface (or posterior surface) 116 configured to cool a surface of an eyeball when the device 110 is positioned underneath the eyelid. In some embodiments, the second surface cools scleral nerves. Directing heat to the underside of an eyelid heats Meibomian glands in the eyelid. Heating Meibomian glands may loosen oils, such as meibum, clogging or partially clogging the glands, so that the glands are unclogged, thereby secreting sufficient oil onto the surface of the eye. Insufficient oil secretion from Meibomian glands is associated with dry eye syndrome. Cooling a surface of the eyeball (using the Peltier device) may stimulate tear production, also to treat dry eye issues, such as aqueous deficient dry eye disease. These and other aspects of underlid devices 110 are explained further herein. As shown in FIG. 1B, in some embodiments, the first surface 114 is convex in order to better align with an adjacent surface of the eyelid 140, and the second surface 116 is concave in order to better align with an adjacent surface of eyeball 150.

The frame 120 may include portions that reside over a person's ear (not shown) to hold the frame 120 in place, and the frame 210 may or may not include glass eyepieces. The frame 120 may be any sort of known eyeglass frame form factor. The frame 120 may be referred to as an external frame because it is worn outside of a human body.

The frame 120 wirelessly communicates with the devices 110, according to an embodiment. For example, the frame 120 is in communication with devices 110 to receive data from one or both devices 110. For example, a device 110 may include one or more sensors to measure variables such as temperature, blink rate, or tear osmolarity, and information collected from measurements may be communicated from a device 110 to frame 120. In an embodiment, the frame 120 also wirelessly charges each device 110 by transmitting power to each device 110, using conventional wireless charging techniques, as discussed further herein.

In an embodiment, the communication device 130 communicates wirelessly with frame 120. For example, the communication device 130 may receive data collected by frame 120, or may transmit operating instructions to frame 120. In an embodiment, the communication device 130 aggregates and/or processes received data and transmits such data or processed data to other devices via a network (not shown), such as the Internet.

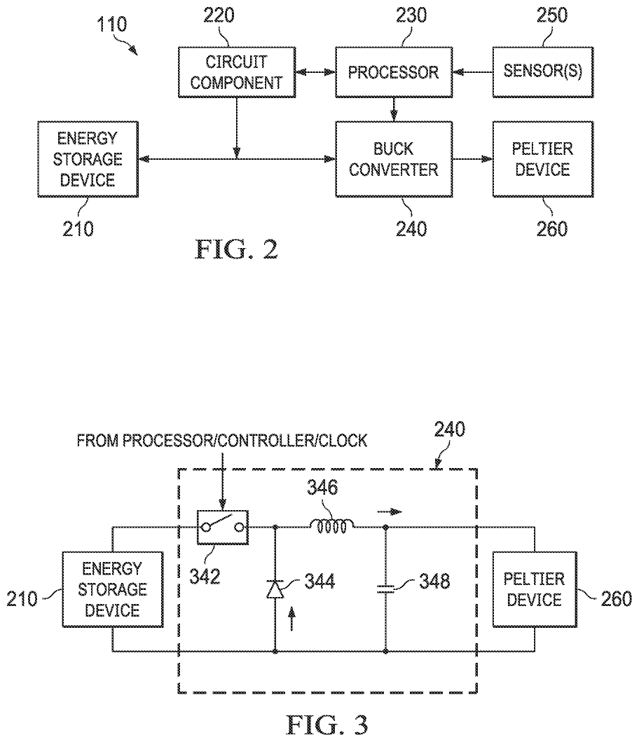

FIG. 2 presents a block diagram of an embodiment of an underlid device 110. In this embodiment, the device 110 includes one or more energy storage devices 210, one or more circuit components 220, such as antennas or coils, configured for wireless charging and communication, a processor 230, a buck converter 240, a Peltier device 260, and one or more sensors 250.

An energy storage device 210 stores energy for powering device 110 (power is understood in the art to be energy delivered per unit time). Examples of an energy storage device 210 include a capacitor, such as a supercapacitor, or a battery. The energy storage device 210 includes two or more supercapacitors, which are connected in series or in parallel, according to an embodiment.

An example circuit component 220 is an antenna, such as a loop antenna. The antenna generally can take any useful form for performing wireless charging via inductive wireless charging and/or for providing communications capability for the device 110. The antenna may reside on a surface of the device 110 or may reside inside the device 110.

Exemplary device 110 in FIG. 2 includes one or more sensors 250 as shown. An example sensor 250 is a pair of electrodes. Such electrodes may be configured to sense the onset of a blink via electromyography (EMG) to provide blink detection. For example, the electrodes may measure electric potential or voltage generated by a conjunctiva or other cells in the eyelid to detect the onset of a blink. The electrodes may also, or alternatively, be configured to stimulate the eyelid muscles to cause a person to blink. Sensors 250 may also include a temperature sensor configured to generate a temperature signal based on a temperature of Meibomian glands. In one embodiment, when the underlid device 110 is placed or mounted underneath an eyelid, such a temperature sensor is positioned to contact a portion of the conjunctiva, adjacent to the Meibomian glands. Sensors 250 may also include an osmolarity sensor configured to generate a moisture signal indicative of an amount of moisture, such as an amount of tear solution, in contact with the sensor. For example, a pair of electrodes can be used to measure tear conductance or impedance, which is used as a measure of tear osmolarity, and the processor 230 may utilize a look-up table or formula to convert conductance measurements to osmolarity values. In an embodiment, the electrodes are formulated from platinum iridium. For example, if a small voltage (AC or DC) is applied across the electrodes, a small current (AC or DC) is generated that can be measured, with the resulting impedance computed as the complex voltage divided by the complex current according to Ohm's law.

As shown, the device 110 in FIG. 2 also includes a processor 230. The processor 230 may take the form of any known processor, such as an integrated circuit (IC), an application-specific integrated circuit (ASIC), a field-programmable gate array (FPGA), or a general-purpose processor. The processor 230 is configured to provide any combination of the following: heating control, power management (such as managing the energy storage element 210 or energy harvesting via wireless charging), EMG blink sensing, blink timing, or stimulating the blink reflex, according to an embodiment. For example, in some embodiments, processor 230 couples to sensors 250 (such as a pair of electrodes) to provide both blink sensing and blink stimulation, taking one or more measurements from electrodes to perform blink sensing and applying voltages or currents to electrodes to perform blink stimulation by stimulating the eyelid.

The device 110 in FIG. 2 also includes a Peltier device 260, sometimes referred to as a Peltier heat pump. A voltage applied across a Peltier device, such as Peltier device 260, causes heat transfer within the device, such that one side of the Peltier device increases in temperature and an opposite side of the Peltier device decreases in temperature, resulting a temperature difference or gradient across the Peltier device.

The Peltier device 260 comprises a first surface (or anterior surface) configured to heat the eyelid when the device is positioned underneath the eyelid. Correspondingly, the Peltier device 260 comprises a second surface (or posterior surface) configured to cool a surface of an eyeball when the device is positioned underneath the eyelid. In some embodiments, the second surface cools scleral nerves. By powering the Peltier device 260, such as using the energy storage device 210 to apply a voltage across Peltier device 260, the Meibomian glands are heated, and the scleral nerves are cooled when the ophthalmic device 110 is mounted or positioned underneath an eyelid of the eye. By heating Meibomian glands, sufficient oils may be produced to relieve dry eye, and cooling scleral nerves can trigger reflex tear production also relieving dry eye. Cooling scleral nerves also can trigger a blink reflex, which coincides with Meibomian gland heating to provide additional assistance in oil flow from Meibomian glands. Thus, the Peltier device 260 is a convenient device for treating Meibomian gland dysfunction and/or aqueous deficient dry eye disease, leading to increased flow of meibum oil and/or increased tear production, according to at least one embodiment.

The device 110 in FIG. 2 also includes buck converter 240. A buck converter, such as buck converter 240, is a type of DC-to-DC power converter. This disclosure recognizes that a buck converter 240 provides relatively high efficiency power delivery to the Peltier device 260. For example, some embodiments achieve 50% efficiency, compared with roughly 1% efficiency when a buck converter is not used. This disclosure recognizes that a buck converter 240 delivers sufficient power for operating the device 110, despite sometimes producing undesirable voltage ripple, and recognizes that the resulting device 110 operates sufficiently. Accordingly, this disclosure recognizes that a power supply that includes one or more supercapacitors connected in series with a buck converter supplies sufficient power to the Peltier device 260 and other components to operate the device 110, according to at least one embodiment. More generally, some embodiments utilize a known circuit coupled between the Peltier device 260 and energy storage device 210 for delivering power from a DC power source to a Peltier device. In some embodiments, such a circuit includes a buck converter.

An example embodiment of a buck converter 240 is illustrated in FIG. 3. In this embodiment, the buck converter includes a switch 342 (such as one or more transistors), a diode 344, an inductor 346, and a capacitor 348 configured as shown in FIG. 3. The buck converter 240 operates in an “on-state” when switch 342 is closed and in an “off-state” when switch 342 is open. As shown in FIG. 3, the buck converter 240 is connected between the energy storage device 210 and Peltier device 260. In some embodiments, the energy storage device 210 and Peltier device 260 together form all or part of a power supply.

A processor or controller, such as processor 230, controls the operation of the switch 342, according to one embodiment. In some embodiments, the switch is closed periodically for a duration of time and then opened at the end of the duration of time, and the process is repeated. Both the period and the duration of time may be adjustable to adjust the frequency of operation (computed as inverse of the period), periodicity and/or duty cycle. For example, the switch 342 may be closed for a half cycle and opened for a half cycle, wherein the cycle repeats at a frequency of several or tens or hundreds of kilohertz (kHz).

According to some experiments, 50 milliwatts (mW) of power may be used to power the underlid device 110. Operating at 50 mW for is requires 50 millijoules (mJ) of energy. Energy stored in a capacitor can be represented as 0.5*C*V2, where C represents capacitance and V represents voltage. As an example, a 7.5 millifarad (mF) supercapacitor, as an example energy storage device 210, operating at 2.6V with a 25Ω source impedance provides a small form factor. A DC-to-DC power converter, such as a buck converter, must be able to deliver 50 mW into a 0.3Ω load, according to some embodiments.

According to some embodiments, a buck converter 240 using a 30 microhenry (uH) inductor 346 that is 1 mm×0.5 mm×6 mm, switching at 100 kHz (using switch 342), is both desirable and realizable. Such a device may be about 50% efficient (limited by supercapacitor source resistance and other losses). This is much better than alternative embodiments that achieve only 1% efficiency when a buck converter is not used.

In some embodiments, wireless charging of a device 110, for example using a wireless charger in frame 120, can occur at a 20 microampere (μA) rate. A 20 μA rate of charging would take roughly 16 mins to recharge a 2.6V, 7.5 mF capacitor. It is possible to boost charging past 20 uA (e.g., up to 100 μA), which would increase the rate of charging at the expense of higher cost or complexity. Wireless charging of the device 110 may occur while power is being delivered to a Peltier device 260, according to some embodiments.

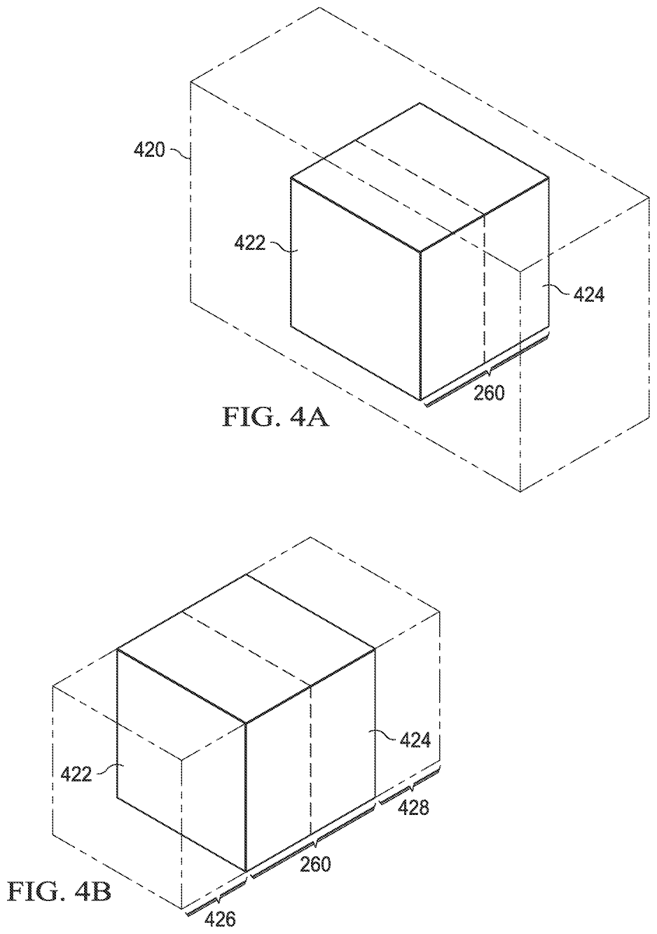

FIGS. 4A and 4B present perspective views of portions of different embodiments of underlid device 110 that include a Peltier device 260. In operation, a voltage is applied across Peltier device 260 which results in heat transfer from one portion of the Peltier device 260 to another. Referring to FIGS. 4A and 4B, when a voltage is applied across the Peltier device 260, one surface 422 increases in temperature and another surface 424 decreases in temperature as heat transfer occurs within the device 260. There is thus a temperature gradient produced across the Peltier device 260, between a higher temperature surface 422 to a lower temperature surface 424.

The Peltier device 260 in FIG. 4A is surrounded by a thermal insulator 420, such as silicone, that helps restrict unwanted loss of heat from the Peltier device 260, such as through heat transfer by radiation from the device 260 or conduction from the device 260 to surrounding materials. In use, the surface 422 is positioned proximate an eyelid and the surface 424 is positioned proximate an eyeball. When electric power is supplied to the Peltier device 260 to cause heat transfer, the surface 422 is used to heat Meibomian glands in the eyelid, thereby loosening oils that have solidified and blocked or partially blocked Meibomian glands, and the surface 424 is used to cool scleral nerves in the eyeball, which can have the effect of inducing tear production, thereby treating dry eye by replacing evaporated tear solution.

The Peltier device 260 FIG. 4B is not necessarily surrounded by a thermal insulator but rather surfaces 422 and 424 are adjacent to, with at least a portion resting against, thermal conductors 426 and 428, respectively. An example thermal conductor is a hydrogel, which is also comfortable for patients. In use, the surface 422 is positioned proximate an eyelid and the surface 424 is positioned proximate an eyeball. When voltage is applied to the Peltier device 260 to cause heat transfer, the surface 422 is used to heat Meibomian glands in the eyelid, as the thermal conductor 426 facilitates heat transfer from surface 422 to the eyelid, and the surface 424 is used to cool scleral nerves in the eyeball, as the thermal conductor 428 facilitates heat transfer from the eyeball to surface 424. A thermal conductor is a material generally understood to provide relatively high thermal conductivity, such as thermal conductivity exceeding 100 W/m/K (Watts/meter/Kelvin) at around 293 K and atmospheric pressure. A thermal insulator is a material generally understood to provide relatively low thermal conductivity, such as thermal conductivity below 1 W/m/K at the conditions described above.

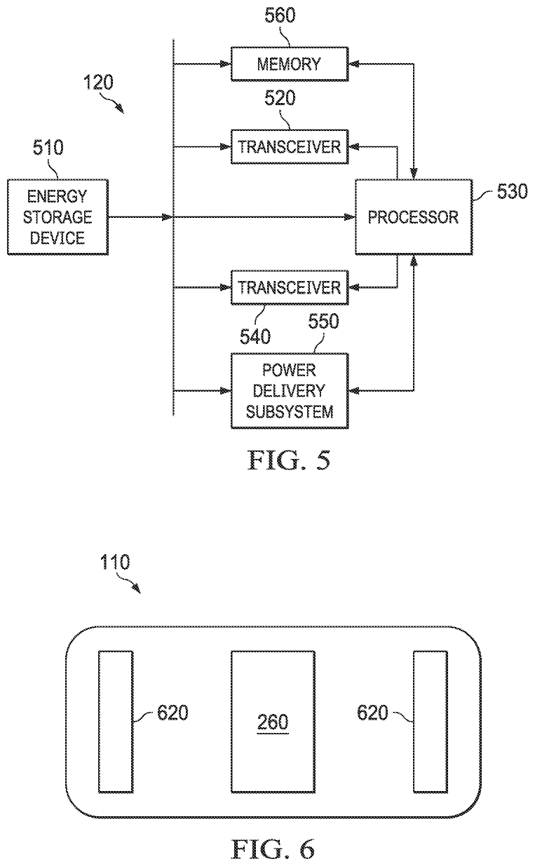

FIG. 5 is a block diagram of components in a frame 120, according to an embodiment. In this embodiment, frame 120 includes energy source 510, a power delivery subsystem 550, a processor 530, communication transceivers 520 and 540, and a memory 560. The energy source 510 provides energy for the other components in the frame 120, and a battery or one or more capacitors are examples of energy source 510.

The energy source 510 may also provide energy to power the underlid device 110. The power delivery subsystem 550 allows power to be delivered from frame 120 to at least one underlid device 110, such as via wireless charging. For example, the power delivery subsystem 550 includes one or more coils (sometimes referred to herein as wireless charging coils) for electromagnetic coupling with one or more coils in an underlid device 110 to perform inductive wireless charging, according to an embodiment.

The communication transceiver 520 performs communication between the frame 120 and underlid device 110. For example, the communication transceiver 520 receives data collected in an underlid device 110. The data collected in the underlid device 110 may include temperature, osmolarity, and/or blink rate measurements. The communication transceiver 520 may utilize near-field communications (NFC), radio-frequency identification (RFID), or Bluetooth Low Energy (BLE) as examples.

The communication transceiver 540 performs communication between the frame 120 and a communication device 130, such as a cell phone or smart phone. For example, if the frame 120 receives data collected in an underlid device 110, the frame 120 can use communication transceiver 540 to send the collected data to the communication device 130, so that the data may ultimately be shared with a physician or other health care provider. As another example, the frame 120 may receive instructions for operation of the underlid device 110 from a physician or other health care provider.

The processor 530 in FIG. 5 is configured to control the operation of communication transceivers 520 and 540 and power delivery subsystem 550. The memory 560 is a semiconductor memory used to store data and/or instructions for other components. The memory 560 is any suitable semiconductor memory, such as a random-access memory (RAM) (such as a synchronous dynamic RAM or SDRAM), a read-only memory (ROM) (such as a programmable ROM or PROM), a flash memory or any combination thereof. The memory 560 may be used to store the instructions for operating processor 530, and/or transceivers 520 and 540. The processor 530 is any suitable processor, such as an integrated circuit (IC), a field-programmable gate array (FPGA), digital signal processor (DSP), or general-purpose processor.

Generally, any creation, storage, processing, and/or exchange of user data associated with the method, apparatus, and/or system disclosed herein is configured to comply with a variety of privacy settings and security protocols and prevailing data regulations, consistent with treating confidentiality and integrity of user data as an important matter. For example, the apparatus and/or the system, such as device 110, frame 120, and/or system 100, may include a module that implements information security controls to comply with a number of standards and/or other agreements. In some embodiments, the module receives a privacy setting selection from the user and implements controls to comply with the selected privacy setting. In other embodiments, the module identifies data that is considered sensitive, encrypts data according to any appropriate and well-known method in the art, replaces sensitive data with codes to pseudonymize the data, and otherwise ensures compliance with selected privacy settings and data security requirements and regulations.

FIG. 6 is a planar view of an eyelid side of an underlid device 110, according to one embodiment. The underlid device 110 in FIG. 6 includes a Peltier device 260, as described herein, and electrodes 620. The electrodes 620 may be used for blink detection. For example, the electrodes 620 may be configured to sense the onset of a blink via electromyography (EMG) to provide blink detection. The electrodes 620 are examples of sensors 250 presented in the embodiment in FIG. 2. In an embodiment, a portion of the surface of each of the electrodes 620 extends to the outer surface of the device 110. Likewise, in an embodiment, a portion of the surface of the Peltier device 260 extends to the outer surface of the device 110. Alternatively, the device 110 is covered by an overmold, such as using a silicone elastomer, suitable for contacting surfaces of the eye and eyelids, and the electrodes 620 and/or Peltier device 260 are located entirely beneath the surface of the overmold.

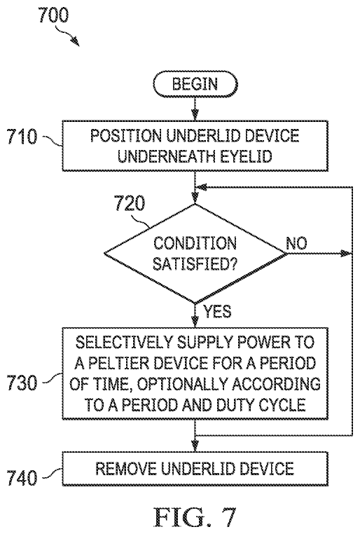

FIG. 7 presents a method 700 of operating an underlid device, such as the embodiments of underlid devices 110 described previously. The method 700 commences in step 710. In step 710, an underlid device, such as the embodiments of underlid devices 110 described previously, is positioned underneath an eyelid. For example, an underlid device is placed between an eyelid and eyeball as shown in FIG. 1B. Once positioned, in step 720 a determination is made whether a condition is satisfied. In one embodiment, sensors, such as sensors 250 described previously, are used to provide information to a processor, such as processor 230, in the underlid device, for use in determining whether the condition is satisfied.

Based on sensor readings, the processor determines whether a condition is satisfied. According to one embodiment, the sensors may include a pair of electrodes, and the electrodes are used to measure tear film conductance or impedance, which can be used to provide a measure of tear osmolarity as discussed herein. In one embodiment, if the tear osmolarity exceeds a threshold, thereby signaling a dry eye condition, the condition in step 720 is satisfied and a electrical power is supplied to a Peltier device to apply a heated surface of the underlid device to Meibomian glands and a cooled surface to the surface of the eye, thereby providing relief to or treating the dry eye condition. In one embodiment, a temperature sensor may be included and used to provide a measurement of temperature of an area on or near a surface of the patient underneath the eyelid, and, if the temperature exceeds a threshold, the Peltier devices will not be activated. Thus, a temperature sensor can be used to protect the eyelid from excessive heating.

The same or a different pair of electrodes can be used as a sensor for blink detection, as discussed herein. By detecting blinks and keeping track of the time between blinks, the underlid device can compute a blink rate. The blink rate may be used as a condition in step 720. For example, if the blink rate is too low, the method 700 moves to step 730 in which power is supplied to a Peltier device in the underlid device to stimulate blinking.

In step 730, power is supplied to a Peltier device to activate the Peltier device. The Peltier device may be activated according to a period and duty cycle. For example, the period may be one minute, and the duty cycle may be one percent, such that the Peltier device is activated one percent of each minute for some duration of time. The period and duty cycle may be programmable parameters stored in an underlid device. After the period of time in step 730, the state of the device returns to step 720 to check whether a condition is satisfied and the process repeats. Power may be supplied to the Peltier device using a buck converter, e.g., by periodically opening and closing the switch in a Peltier device at a given frequency, such as 100 kHz, resulting in continuous power delivery.

During the method 700, a frame is worn by the patient, such as the frame 120 described with respect to FIGS. 1A and 5, according to an embodiment. In an embodiment, the frame includes a larger energy source that is used to supply energy to the underlid devices, e.g., through wireless charging as discussed herein. In another embodiment, a periocular ring worn on the eye can be used to supply energy to the underlid device. In such an embodiment, the periocular ring includes a coil and energy storage component for wirelessly charging the underlid device.

Generally, any creation, storage, processing, and/or exchange of user data associated with the method, apparatus, and/or system disclosed herein is configured to comply with a variety of privacy settings and security protocols and prevailing data regulations, consistent with treating confidentiality and integrity of user data as an important matter. For example, the apparatus and/or the system may include a module that implements information security controls to comply with a number of standards and/or other agreements. In some embodiments, the module receives a privacy setting selection from the user and implements controls to comply with the selected privacy setting. In other embodiments, the module identifies data that is considered sensitive, encrypts data according to any appropriate and well-known method in the art, replaces sensitive data with codes to pseudonymize the data, and otherwise ensures compliance with selected privacy settings and data security requirements and regulations.

Persons skilled in the art will recognize that the devices, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A system for treating dry eye comprising:
an underlid device comprising an anterior surface and a posterior surface, wherein the anterior surface is configured to contact a portion of an eyelid, and wherein the posterior surface is configured to contact a portion of an eyeball, the underlid device further comprising:
a Peltier heat pump, the Peltier heat pump comprising:
a first surface configured to heat the eyelid when the underlid device is positioned between the eyelid and the eyeball; and
a second surface configured to cool a portion of a surface of the eyeball when the underlid device is positioned between the eyelid and the eyeball; and
an energy storage element coupled to the Peltier heat pump and configured to supply power to the Peltier heat pump.

2. The system of claim 1, further comprising a buck converter configured to couple the energy storage element to the Peltier heat pump, wherein the buck converter comprises a diode connected in parallel with a capacitor, wherein the capacitor is connected in parallel with the Peltier heat pump, and wherein the buck converter further comprises an inductor connected between the diode and the capacitor.

3. The system of claim 2, wherein the underlid device further comprises a processor, wherein the buck converter further comprises a switch, and wherein the processor is coupled to the switch and the processor is configured to control the switch to deliver power from the energy storage element to the Peltier heat pump.

4. The system of claim 1 further comprising a frame configured to be worn by a patient, the frame comprising:
a second energy storage element; and
an inductor coupled to the second energy storage element, wherein the inductor is configured to wirelessly couple to the underlid device to charge the energy storage element.

5. The system of claim 1, further comprising a buck converter configured to couple the energy storage element to the Peltier heat pump, wherein the energy storage element comprises at least one capacitor.

6. The system of claim 3, further comprising a pair of electrodes configured to generate a signal to indicate tear film impedance, wherein the processor is configured to receive the signal and, based on the signal, control the buck converter to supply power to the Peltier heat pump.

7. The system of claim 1, wherein the underlid device further comprises a layer of thermally conducting material forming a portion of the anterior surface, wherein the layer of thermally conducting material conducts heat from the first surface to heat the eyelid.

8. The system of claim 1, wherein the Peltier heat pump is surrounded by a thermally insulating material on all sides except the first surface and the second surface.

9. A device for treating dry eye and configured to be positioned between an eyelid and sclera of an eyeball, the device having a first surface that is convex for contacting the eyelid and having a second surface that is concave for contacting the sclera, the device comprising:
an energy storage device;
a Peltier device coupled to the energy storage device, the Peltier device comprising a third surface configured to heat a portion of the eyelid and a fourth surface configured to cool a portion of the sclera when power is delivered to the Peltier device; and
a circuit configured to couple the energy storage device to the Peltier device to supply power to the Peltier device, wherein heat is transferred within the Peltier device when power is supplied to the Peltier device resulting in heating of the third surface and cooling of the fourth surface, thereby heating the portion of the eyelid and cooling the portion of the sclera, respectively.

10. The device of claim 9, wherein the energy storage device comprises one or more supercapacitors.

11. The device of claim 10, wherein the circuit comprises a buck converter, the device further comprising:
a sensor configured to generate a signal;
and a processor coupled to the buck converter and configured to receive the signal, wherein the processor is configured to activate the buck converter to supply power to the Peltier device based on the signal.

12. The device of claim 9, wherein the device is configured to cover only a portion of the sclera and not cover any portion of a pupil of the eyeball.

13. The device of claim 9, further comprising a wireless charging coil coupled to the energy storage device, wherein the wireless charging coil is configured to receive power wirelessly from a device worn by a patent and charge the energy storage device.

14. The device of claim 9, wherein the circuit comprises a buck converter, wherein the buck converter comprises a switch connected in series between the energy storage device and the Peltier device, and wherein the switch is configured to periodically close and open according to a frequency to deliver power to the Peltier device thereby heating the third surface and cooling the fourth surface.

15. The device of claim 11, wherein the sensor comprises a pair of electrodes, wherein the electrodes generate the signal, and wherein the signal is used by the processor to detect a blink or to measure tear film impedance.

16. The device of claim 13, wherein the circuit comprises a buck converter, and wherein the buck converter further comprises a diode, an inductor, and a capacitor, and wherein the diode, the capacitor, and the Peltier device are connected in parallel.

17. A method of ophthalmic treatment using an underlid device when the underlid device is positioned beneath the surface of an eyelid, wherein the underlid device comprises an energy storage element coupled to a Peltier device, wherein the Peltier device comprises a first surface and a second surface, wherein the first surface is configured to heat a portion of a conjunctiva of the eyelid and the second surface is configured to cool a portion of a sclera, wherein the Peltier device is configured to transfer heat such that the first surface increases in temperature and the second surface decreases in temperature when power is supplied using the energy storage element, the method comprising:
generating a signal, by a sensor;
determining that a condition is satisfied based on the signal; and
based on the condition being satisfied, supplying power to the Peltier device using the energy storage element to heat the first surface and cool the second surface, thereby heating the portion of the conjunctiva and cooling the portion of the sclera.

18. The method of claim 17, wherein the sensor comprises a pair of electrodes, and wherein determining that the condition is satisfied comprises using the electrodes to measure tear film impedance.

19. The method of claim 17, wherein the sensor comprises a pair of electrodes, and wherein determining that the condition is satisfied comprises using the electrodes to detect an onset of a blink via electromyography.

20. The method of claim 17, further comprising receiving electric power, using wireless charging, from a frame worn by a patient.
